(19) United States
(12) Plant Patent Application Publication
(10) Pub. No.: US 2003/0051281 P1
(43) Pub. Date: Mar. 13, 2003

(54) Red Filli

(76) Inventors: David W. Fleming, Lincoln, NE (US); Gretchen A. Fleming, Lincoln, NE (US)

Correspondence Address: David Fleming and Gretchen Zwetzig, Fleming's Flower Fields Inc., 8101 S. 14th St., Lincoln, NE, 68512, US

(21) Appl. No.: 09/950,235

(22) Filed: Sep. 10, 2001

(51) Int. Cl. A01H 5/00

(52) U.S. Cl. PLT/252

(57) ABSTRACT

A new and distinct cultivar of winter hardy Lagerstroemia named ‘Red Filli’ is the result of a unique hybridization. This new and distinct cultivar is characterized primarily as to novelty by its extreme cold hardiness to Zone 4, its numerous sturdy bright-red blooms, its compact spreading performance with heavy branching, its vigorous and uniform growth habit, and its floriferous nature from midsummer until frost.

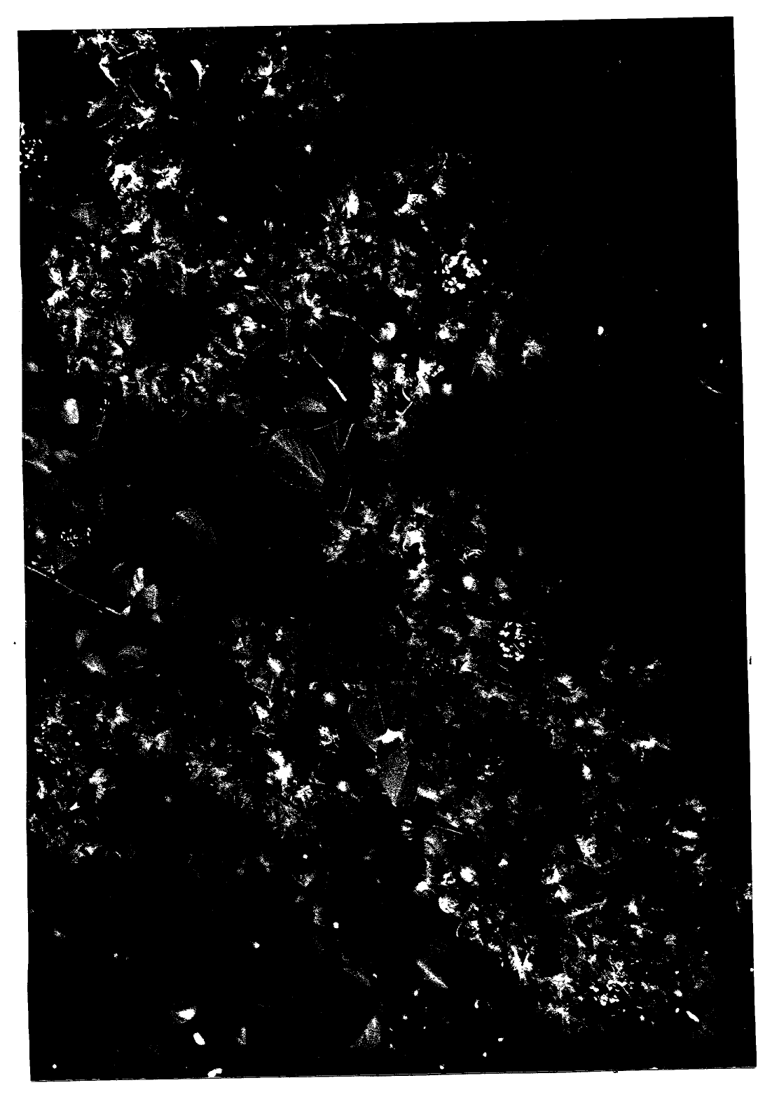

### BACKGROUND OF THE PLANT

The new plant of this invention is the result of a unique hybridization, with the breeding achievement being evidenced in the outstanding combination of characteristics exhibited by this new and distinct Crape Myrtle plant, which include:

(a) The plant being very floriferous with flowers that are outstanding for their wide bright-red blooms.

(b) The plant being dwarf, but vigorous, with compact and uniform spreading action which gives it unique landscape utility; and

(c) The plant being so hardy that it can consistently withstand winter temperatures of at least −30 degrees Fahrenheit.

### SUMMARY OF THE INVENTION

‘Red Filli’ was the result of a 40-year breeding program. Its ancestry includes various seedlings of Lagerstroemia indica More specifically, the plant resulted as a selected hardier seedling from the Fleming Crape Myrtle ‘Filligree’ series. The seedling which most nearly met all above standards was selected.

This new plant first bloomed in the summer of 1978 and was selected by David Fleming and Gretchen Zwetzig on David Fleming's property in Lincoln, NE. Asexual propagation of the plant by cuttings and root division in Lincoln, NE, has shown that the unique and distinguishing features of the plant are faithfully transmitted from generation to generation and appear to be fixed.

Since its origin, the plant has bloomed from midsummer until frost, while exhibiting the above-mentioned distinctive characteristics. This hardy Crape Myrtle plant contributes to the market with its sheer beauty, its compact growth habit, its great resistance to disease and insects, its stability through extremes in rain and drought, and its extreme hardiness.

### BRIEF DESCRIPTION OF THE DRAWINGS

The flower, bud, foliage, and growth habit of the plant are shown in the attached illustrations. More specifically, Sheet 1 shows a close-up of the bloom and foliage. Sheet 2 depicts the growth habit of the cultivar, from the ground up to the apex of the plant. The colors are as true as is reasonably possible to attain in photographic illustrations of this type. The colors illustrated may be slightly off due to light reflectance.

### DESCRIPTION OF THE NEW PLANT

What follows is a detailed description of the new cultivar. The specific color designations set forth by PLATE and number designations are in accordance with the Dictionary of Color (Maerz and Paul), while general color recitations are consistent with ordinary American color terminology. ‘Red Filli’ has not been observed under all possible environmental conditions. It is to be understood that the phenotype may vary significantly with variatios in the environment such as temperature, light intensity, and day length, without, however any difference in genotype of the plant. The following botanical characteristics and observations are taken from the plant when grown under normal outdoor conditions in Lincoln, NE.

Plant:

Tree performance.—From spreading to flat spreading.

Tree form.—Flat globe.

Tree height.—Dwarf, about 1½ feet.

Stem:

Type of trunk.—Bush.

Trunk color.—Brown.

Young bud color.—Cherry red.

Branches.—Color: Brown. Thickness: 3 cm. Branching habit: Heavy; 50 branches in average on main trunk. Internode length: About ½ inch, 1 cm. Average leaf length: About 1½ inches, 3¼ cm. Average leaf width: About ¾ inch, 2 cm.

Foliage:

Leaves (Lamina).—Compact, lanceolate-shaped with acute apex and base.

Margin.—Entire.

Color immature.—Slightly darker than 5-L, page 69, PLATE23.

Color mature.—Same as immature.

Color change.—Purple—“blush”.

Luster.—Dull sheen.

Pet ole.—Green.

Length.—1 cm.

Flower:

Pedicel.—0.5 cm

Calyx tube.—Developed.

Bud.—Rotund.

Cluster.—Conic, broad.

Inflorescence.—numerous, 10 on average per cluster.

Number of flowers.—50 on average per inflorescence.

Shape or form.—Single, six petals.

Fragrance.—Slight.

Petals:

Color.—Slightly lighter than 1-L, page 113, PLATE 45.

Shape.—Orbiculate with a strong wave.

Bloom diameter.—1¾ inches, 4½ cm.

Petal size.—(width of individual petal) ¾ inch, 1½ cm.

Persistence of individual bloom.—2 days.

Reproductive organs:

Style.—Pale-red.

Pistil.—One.

Filaments.—Light red.

Anthers.—Yellow.

Stamens.—30 to 35 arranged around the pistil.

Fruit:

Shape.—Rotund; apex lcm in width.

Color.—Green maturing to burgundy.

Growth:

Habit.—Dwarf, about 1½ feet.

Blooming period.—July until October.

Hardiness: Hardy to at least −30 degrees Fahrenheit.

General Health: plant is very disease resistent; very pest resistant; sturdy through excessive drought or water.

### GENERAL OBSERVATIONS

This plant, as a hybridized hardy Lagerstoemia, is valuable to the landscape market for its improvements in different colored, utstanding flowers, refined foliage, compact and uniform breaking action, all-around vigor, and adaption to extreme environments, including the ability to consistently survive winter temperatures of at least −30 degrees Fahrenheit.

1. A new and distinct cultivar of Lagerstroemia plant, as herein shown and described, characterized by its beautiful flowers and foliage, its compact spreading action, its vigor, and hardiness.
